(19) United States
(12) Patent Application Publication
(10) Pub. No.: US 2017/0055939 A1
(43) Pub. Date: Mar. 2, 2017

(54) ULTRASONIC DIAGNOSTIC APPARATUS AND PROGRAM THEREFOR

(71) Applicant: General Electric Company, Schenectady, NY (US)

(72) Inventors: Saika Nagai, Tokyo (JP); Naohisa Kamiyama, Tokyo (JP); Hiroshi Hashimoto, Tokyo (JP)

(21) Appl. No.: 15/245,491

(22) Filed: Aug. 24, 2016

(30) Foreign Application Priority Data
Aug. 28, 2015 (JP) 2015-168515

(51) Int. Cl. A61B 8/08; A61B 8/14; A61B 8/00

(52) U.S. Cl. CPC A61B 8/0841; A61B 8/467; A61B 8/5207; A61B 8/463; A61B 8/5223; A61B 8/14

(57) ABSTRACT

To provide an ultrasonic diagnostic apparatus enabling one to easily know the correspondence between a needle in an ultrasonic image and an actual needle. An ultrasonic diagnostic apparatus 1 comprises: a first position detecting section for detecting information on a position of a needle N inserted into a subject; an operating section 7 for accepting from a user inputs for, for a plurality of needles N inserted into the subject, appending distinguishing information for distinguishing each of the plurality of needles and storing information on a position detected by the first position detecting section for a needle appended with the distinguishing information; a second position detecting section for detecting information on a position of an ultrasound transmission/reception plane; and a display control section for displaying, in the case that a needle N whose information on a position is stored is included in the ultrasound transmission/reception plane, a marker indicating the needle in an ultrasonic image displayed in a display section for the transmission/reception plane in a manner that the needle is distinguishable from another needle based on the distinguishing information.

### CROSS-REFERENCE TO RELATED APPLICATIONS

This application claims priority to Japan patent application number 2015-168515, filed on Aug. 28, 2015, the entirety of which is incorporated herein by reference.

### BACKGROUND

The present invention relates to an ultrasonic diagnostic apparatus and a program therefor enabling one to easily know the correspondence between a needle in an ultrasonic image and an actual needle when a plurality of needles are inserted into a subject.

Sometimes is applied a therapeutic procedure involving burning a lesion by radiofrequency waves from a needle inserted into a subject. Other times, a needle is inserted into a subject for collecting living tissue from the subject.

An ultrasonic diagnostic apparatus is capable of displaying an ultrasonic image of a subject in real time. Therefore, a procedure has been conventionally practiced in the art involving a user confirming by an ultrasonic image whether or not a needle has reached a position to be burnt or a position for taking living tissue (see Patent Document 1, for example).

In some cases, a plurality of needles are inserted into a subject. In such cases, it is sometimes difficult to recognize which one of the needles a needle observed in an ultrasonic image is. Accordingly, for a user to identify which one of actual needles inserted into a subject a needle observed in an ultrasonic image is, an operation of applying micro-vibration to a needle is sometimes practiced. As such, identification of the correspondence between a needle in an ultrasonic image and an actual needle is cumbersome when a plurality of needles are used.

The invention, in one aspect, for solving the aforementioned problem is an ultrasonic diagnostic apparatus characterized in comprising: a first position detecting section for detecting information on a position of a needle inserted into a subject in a specified coordinate system in a three-dimensional (3D) space; an input section for accepting from a user inputs for, for a plurality of needles inserted into said subject, appending distinguishing information for distinguishing each of said plurality of needles and storing information on a position detected by said first position detecting section for a needle appended with said distinguishing information; a second position detecting section for detecting information on a position in said 3D space of an ultrasound transmission/reception plane formed by an ultrasonic probe performing ultrasound transmission/reception to/from said subject; and a display control section for displaying, in the case that a needle whose information on a position is stored is included in said ultrasound transmission/reception plane whose information on a position is detected by said second position detecting section, a marker indicating said needle in an ultrasonic image displayed in a display section for said transmission/reception plane in a manner that said needle is distinguishable from another needle based on said distinguishing information.

According to the invention in the aspect described above, for a plurality of needles inserted into a subject, the input section accepts from a user an input for appending distinguishing information for distinguishing each of the plurality of needles and storing information on a position detected by the first position detecting section for a needle appended with the distinguishing information, whereby a marker indicating the needle may be displayed in an ultrasonic image in a manner that the needle is distinguishable from another needle. Thus, even in the case that a plurality of needles are inserted into a subject, one may easily know the correspondence between needles in an ultrasonic image and actual needles.

### BRIEF SUMMARY OF THE INVENTION

An ultrasonic diagnostic apparatus characterized in comprising a first position detecting section for detecting information on a position of a needle inserted into a subject in a specified coordinate system in a three-dimensional (3D) space, an input section for accepting from a user inputs for, for a plurality of needles inserted into said subject, appending distinguishing information for distinguishing each of said plurality of needles and storing information on a position detected by said first position detecting section for a needle appended with said distinguishing information, a second position detecting section for detecting information on a position in said 3D space of an ultrasound transmission/reception plane formed by an ultrasonic probe performing ultrasound transmission/reception to/from said subject, and a display control section for displaying, in the case that a needle whose information on a position is stored is included in said ultrasound transmission/reception plane whose information on a position is detected by said second position detecting section, a marker indicating said needle in an ultrasonic image displayed in a display section for said transmission/reception plane in a manner that said needle is distinguishable from another needle based on said distinguishing information.

In an embodiment, an ultrasonic diagnostic apparatus includes a processor, and an input device, said apparatus being characterized in that: said processor executes a first position detecting function, a second position detecting function, and a display control function by a program, wherein: said first position detecting function is a function of detecting information on a position of a needle inserted into a subject in a specified coordinate system in a three-dimensional (3D) space, said input device is for accepting from a user inputs for, for a plurality of needles inserted into said subject, appending distinguishing information for distinguishing each of said plurality of needles and storing information on a position detected by said first position detecting function for a needle appended with said distinguishing information, said second position detecting function is a function of detecting information on a position in said 3D space of an ultrasound transmission/reception plane formed by an ultrasonic probe performing ultrasound transmission/reception to/from said subject, and said display control function is a function of displaying, in the case that a needle whose information on a position is stored is included in said ultrasound transmission/reception plane whose information on a position is detected by said second position detecting function, a marker indicating said needle in an ultrasonic image displayed in a display section for said transmission/reception plane in a manner that said needle is distinguishable from another needle based on said distinguishing information.

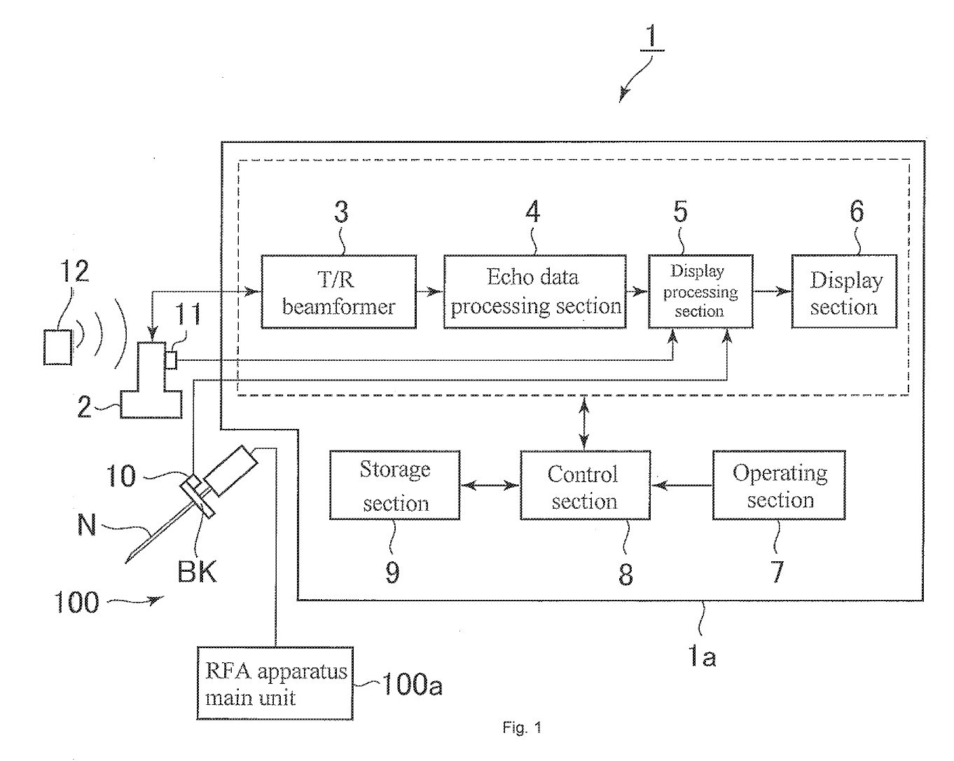

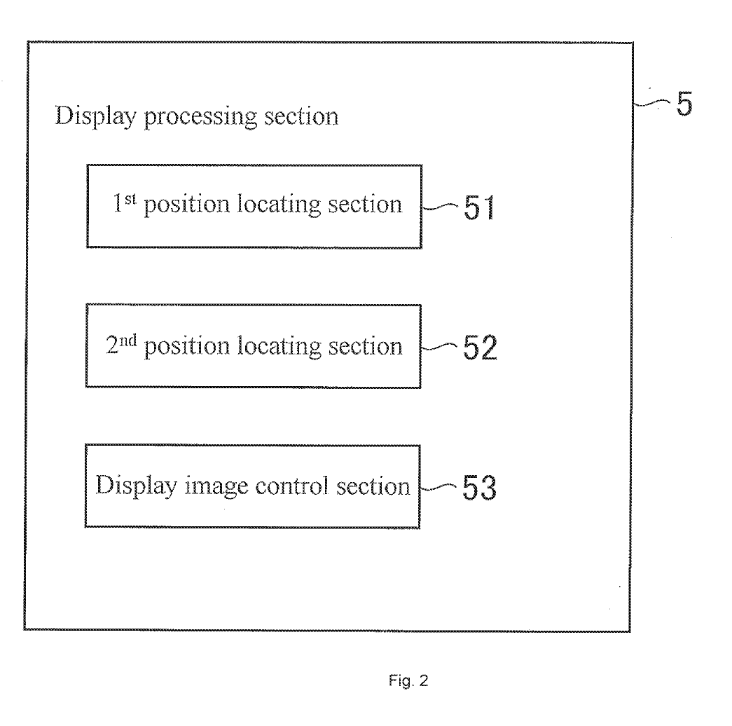

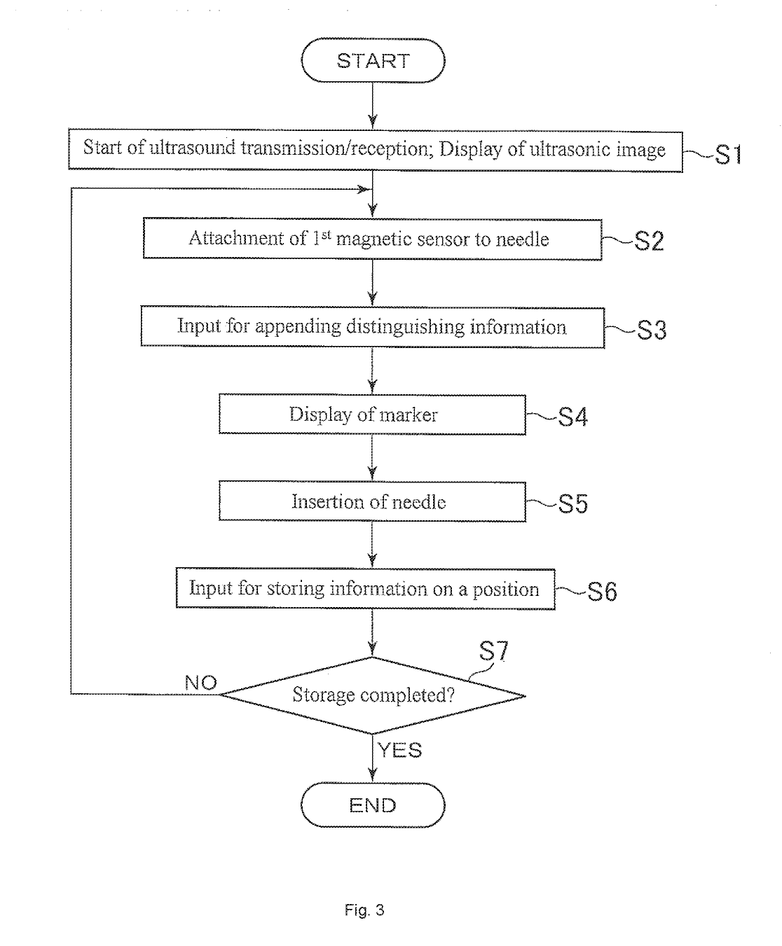

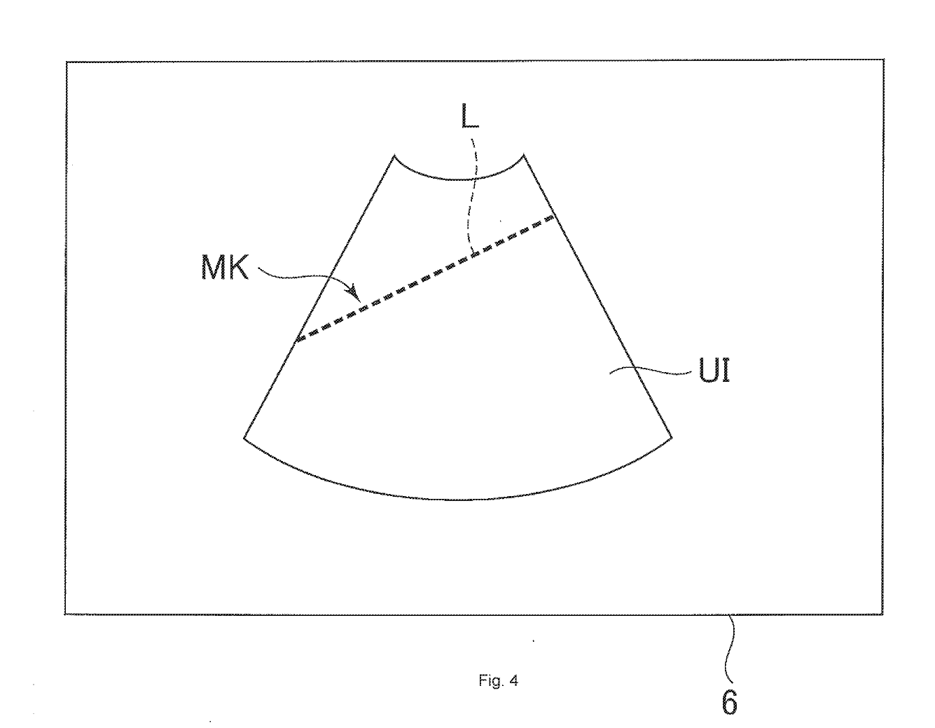

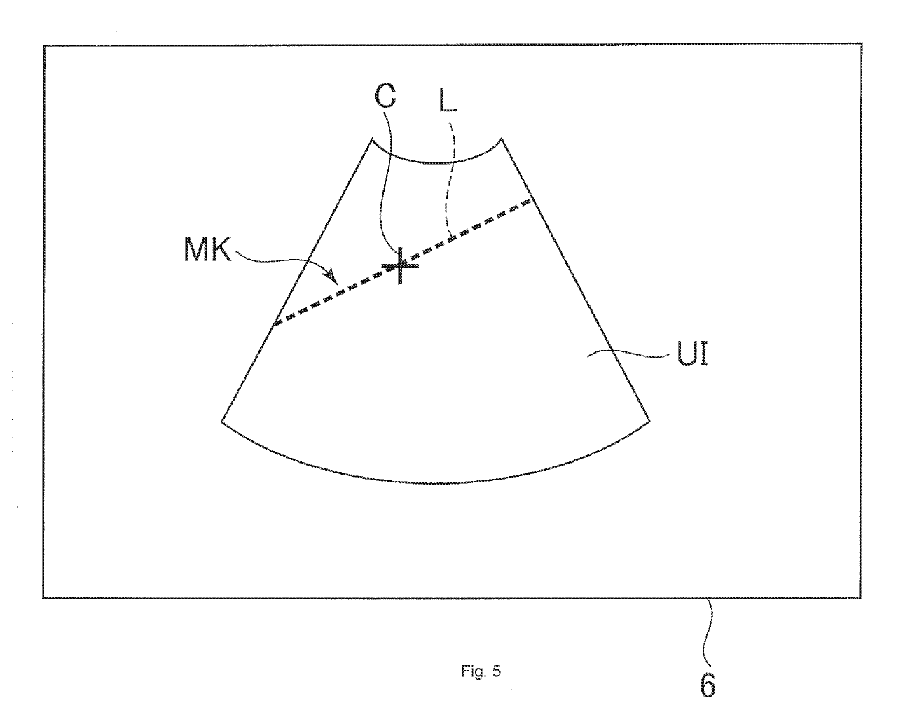

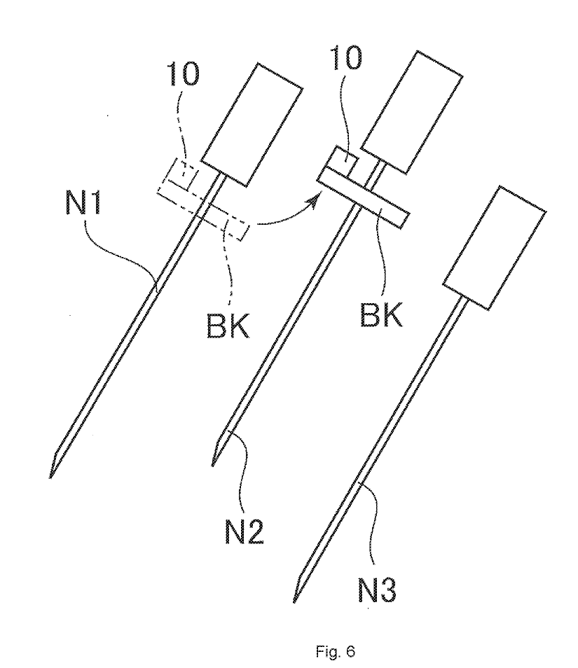

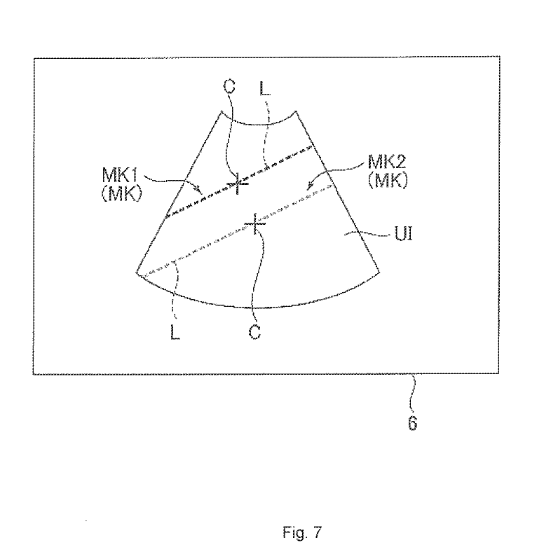

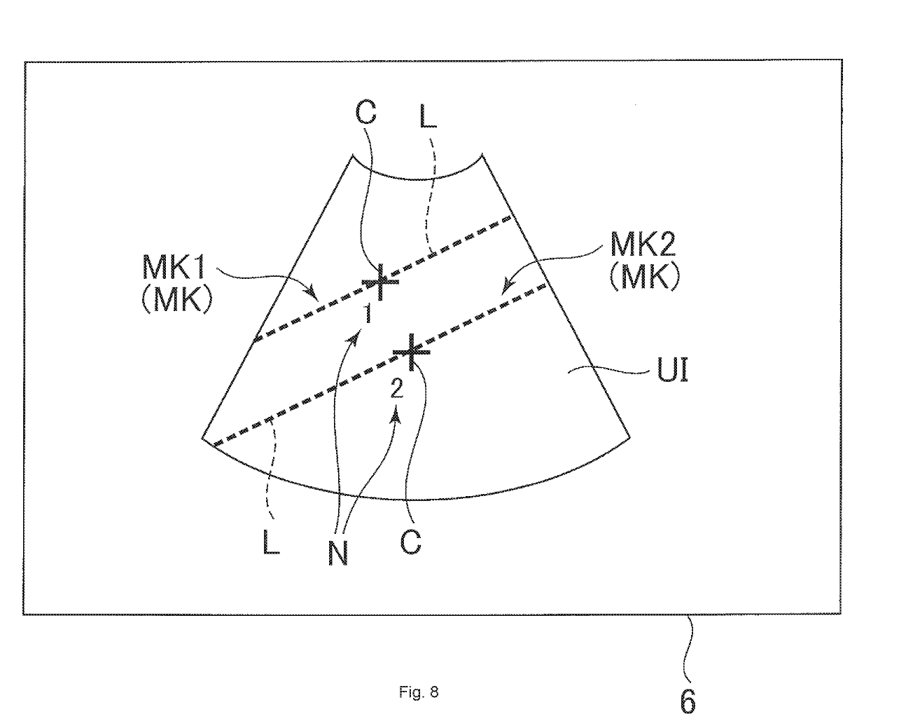

### BRIEF DESCRIPTION OF DRAWINGS

[FIG. 1] A block diagram showing an exemplary general configuration of an ultrasonic diagnostic apparatus in an embodiment of the present invention.

[FIG. 2] A block diagram showing a configuration of the display processing section in the ultrasonic diagnostic apparatus shown in FIG. 1.

[FIG. 3] A flow chart showing the operation of the embodiment.

[FIG. 4] A diagram showing the display section in which an ultrasonic image is displayed with a marker displayed thereon in a condition that no needle is inserted into the subject.

[FIG. 5] A diagram showing the display section in which an ultrasonic image is displayed with a marker displayed therein in a condition that a needle is inserted into the subject.

[FIG. 6]A diagram explaining removal and attachment of a first magnetic sensor.

[FIG. 7] A diagram showing the display section in which an ultrasonic image is displayed with markers displayed therein.

[FIG. 8] A diagram showing the display section in which numeric characters are displayed along with the markers.

Now an embodiment of the present invention will be described. An ultrasonic diagnostic apparatus 1 shown in FIG. 1 comprises an ultrasonic probe 2, a transmission/reception (T/R) beamformer 3, an echo data processing section 4, a display processing section 5, a display section 6, an operating section 7, a control section 8, and a storage section 9. The ultrasonic diagnostic apparatus 1 has a configuration as a computer.

The T/R beamformer 3, echo data processing section 4, display processing section 5, display section 6, operating section 7, control section 8, and storage section 9 are provided in an apparatus main unit la of the ultrasonic diagnostic apparatus 1. The apparatus main unit la and the ultrasonic probe 2 are connected with each other via a cable. The apparatus main unit la is also connected via a cable with a first magnetic sensor 10 attached with a needle N.

The ultrasonic probe 2 is configured to have a plurality of ultrasonic vibrators (not shown) arranged in an array, and by the ultrasonic vibrators, ultrasound is transmitted to a subject and echo signals thereof are received. The ultrasonic probe 2 represents an exemplary embodiment of the ultrasonic probe in the present invention.

The ultrasonic probe 2 is provided with a second magnetic sensor 11 constructed from a Hall element, for example. By the second magnetic sensor 11, magnetism generated from a magnetism generating section 12 constructed from a magnetism generating coil, for example, is detected. By the magnetism generated from the magnetism generating section 12, a coordinate system is formed in a 3D space. The coordinate system is one having its origin lying at the magnetism generating section 12, and represents an exemplary embodiment of the specified coordinate system in the present invention.

A detected signal at the second magnetic sensor 11 is directed to be input to the display processing section 5. The detected signal at the second magnetic sensor 11 may be input to the display processing section 5 via a cable, which is not shown, or it may be wirelessly input to the display processing section 5. The magnetism generating section 12 and second magnetic sensor 11 are provided for detecting a position and an orientation of the ultrasonic probe 2 and a position of an ultrasound transmission/reception plane in the coordinate system in the 3D space, which will be described below.

The T/R beamformer 3 supplies an electric signal to the ultrasonic probe 2 for transmitting ultrasound in specified scan conditions from the ultrasonic probe 2 based on a control signal from the control section 8. The T/R beamformer 3 also performs signal processing, such as A/D conversion and phased addition processing, on echo signals received by the ultrasonic probe 2, and outputs signal-processed echo data to the echo data processing section 4.

The echo data processing section 4 performs processing for producing an ultrasonic image on the echo data output from the T/R beamformer 3. For example, the echo data processing section 4 performs B-mode processing, including logarithmic compression processing and envelope detection processing, to generate B-mode data.

The display processing section 5 has a first position locating section 51, a second position locating section 52, and a display image control section 53, as shown in FIG. 2. The first position locating section 51 detects information on a position of the needle N inserted into the subject in the coordinate system in the 3D space having its origin lying at the magnetism generating section 12. More specifically, the needle N is provided with the first magnetic sensor 10 constructed from a Hall element, for example, via a bracket BK. The first magnetic sensor 10 is removably attached to the needle.

By the first magnetic sensor 10, magnetism generated from the magnetism generating section 12 is detected. The detected signal at the first magnetic sensor 10 is input to the display processing section 5. The first position locating section 51 first locates coordinates of the position of the first magnetic sensor 10 in the coordinate system in the 3D space having its origin lying at the magnetism generating section 12 based on the magnetism-detecting signal from the magnetic sensor 10. The first position locating section 51 then locates coordinates of the position of the needle N attached with the first magnetic sensor 10 based on the coordinates of the position of the first magnetic sensor 10, a positional relationship between the needle N from its one end to the other end and the first magnetic sensor 10, and the length of the needle N. The first position locating section 51 may also locate coordinates of the position of an extension line of the needle N attached with the first magnetic sensor 10. The first position locating section 51 and first magnetic sensor 10 represent an exemplary embodiment of the first position detecting section in the present invention.

The second position locating section 52 locates information on a position of an ultrasound transmission/reception plane formed by the ultrasonic probe 2. The information on a position is the information on a position in the 3D space having its origin lying at the magnetism generating section 12. More particularly, the second position locating section 52 first locates coordinates of the position and orientation of the ultrasonic probe 2 in the coordinate system in the 3D space having its origin lying at the magnetism generating section 12 based on a magnetism-detecting signal from the second magnetic sensor 11. The second position locating section 52 then locates coordinates of the position of the ultrasound transmission/reception plane formed by the ultrasonic probe 2 in the coordinate system in the 3D space based on the coordinates of the position and orientation of the ultrasonic probe 2. The second position locating section 52 and second magnetic sensor 11 represent an exemplary embodiment of the second position detecting section in the present invention.

It should be noted here that the needle N is a needle used in radiofrequency ablation (RFA), for example, and emits radiofrequency waves. In the present example, the ablation with radiofrequency waves is achieved by a plurality of the needles N. The needles N are connected with an RFA apparatus main unit 100a. The needles N and RFA apparatus main unit 100a constitute an RFA apparatus 100. It is by the RFA apparatus main unit 100a that emission of radiofrequency waves by the needles N is controlled.

The display image control section 53 scan-converts data input from the echo data processing section 4 by a scan converter to generate ultrasonic image data. The display image control section 53 then displays an ultrasonic image based on the ultrasonic image data in the display section 6. For example, the display image control section 53 scan-converts B-mode data to generate B-mode image data, and displays a two-dimensional B-mode image in the displays display section 6.

The display image control section 53 also displays a marker MK (see FIG. 5) indicating a needle N in an ultrasonic image, such as a B-mode image, displayed in the display section 6. Details thereof will be discussed later. The display image control section 53 represents an exemplary embodiment of the display control section in the present invention.

The display section 6 is an LCD (Liquid Crystal Display) or an organic EL (Electro-Luminescence) display. The display section 6 represents an exemplary embodiment of the display section in the present invention.

The operating section 7 is configured to comprise an input device, such as a keyboard for accepting a command and/or information from the user, and a pointing device such as a button and a trackball, etc., although not particularly shown. For example, the operating section 7 accepts from the user an input for, for a plurality of needles N inserted into the subject, appending distinguishing information for distinguishing each of the plurality of needles N and storing information on a position located by the first position locating section 51 of the needle N appended with the distinguishing information. In the present example, the distinguishing information is information on a color different from each other set for each of a plurality of markers MK. The distinguishing information appended in response to the input at the operating section 7 is stored in the storage section 9 along with the information on a position of the needle N appended with the distinguishing information. The operating section 7 represents an exemplary embodiment of the input section in the present invention.

The control section 8 is a processor such as a CPU (Central Processing Unit). The control section 8 loads thereon a program stored in the storage section 9 to control each section in the ultrasonic diagnostic apparatus 1. For example, the control section 8 loads thereon programs stored in the storage section 9, and performs functions of the T/R beamformer 3, echo data processing section 4, and display processing section 5 according to the loaded program.

The control section 8 may execute all of the functions of the T/R beamformer 3, all of the functions of the echo data processing section 4, and all of the functions of the display processing section 5 by the programs, or only part of the functions by the programs. In the case that the control section 8 executes only part of the functions, the remaining functions may be executed by hardware such as circuitry.

Additionally, the functions of the T/R beamformer 3, echo data processing section 4, and display processing section 5 may be implemented by hardware such as circuitry.

The storage section 9 is an HDD (Hard Disk Drive), and/or semiconductor memory such as RAM (Random Access Memory) and ROM (Read Only Memory). The ultrasonic diagnostic apparatus 1 may have all of the HDD, RAM, and ROM as the storage section 9. The storage section 9 may also be a portable storage medium, such as a CD (Compact Disk) or a DVD (Digital Versatile Disk).

The programs executed by the control section 8 are stored in a non-transitory storage medium, such as HDD and/or ROM. The programs may be stored in a portable non-transitory storage medium, such as a CD (Compact Disk) or a DVD (Digital Versatile Disk).

The storage section 9 also stores therein the information on a position of the needle N detected by the first position locating section 51 and the distinguishing information appended to the needle N. The information on a position of a needle N is information on a position of the needle N from its one end to the other end. Alternatively, information on positions of the needle N per se and an extension line of the needle N may be stored in the storage section 9 as the information on a position of a needle N. The information on a position of a needle N stored in the storage section 9 also includes information on a position of the needle tip of the needle N.

Moreover, the storage section 9 stores therein a plurality pieces of distinguishing information, for example, with their order of appending to the needles N specified. Since the distinguishing information is information on a color in the present example, information on a plurality of different colors is stored with its order of appending to the needles N specified.

Now processing of appending distinguishing information to a needle and storing information on a position of the needle in the ultrasonic diagnostic apparatus 1 in the present example will be described based on the flow chart in FIG. 3. First, at Step S1, the user puts the ultrasonic probe 2 onto a body surface of the subject, and starts ultrasound transmission/reception. A B-mode image, for example, is thus displayed in the display section 6.

Next, at Step S2, the user attaches the first magnetic sensor 10 to one of the needles N. The present example assumes that three needles N1, N2, N3 are employed, as will be discussed later. Here, the user attaches the first magnetic sensor 10 to the needle N1.

Next, at Step S3, the user inputs a command at the operating section 7 for appending distinguishing information to the needle N1 attached with the first magnetic sensor 10. Upon the input, the control section 8 stores distinguishing information for the needle N1 attached with the first magnetic sensor 10 in the storage section 9. For example, by the user pressing a specified button in the operating section 7 once, the control section 8 stores the distinguishing information for the needle N1 into the storage section 9.

The distinguishing information for the needle N1 is color information for differentiating it from the other needles N2, N3. The color information is appended in the order stored in the storage section 9. For example, in the case that an operation that red should be first appended is stored in the storage section 9, the needle N1 is appended with red.

Next, at Step S4, the display image control section 53 displays a marker MK in an ultrasonic image UI displayed in the display section 6, as shown in FIG. 4. The display image control section 53 displays the marker MK in the same color as that in the color information appended at Step S3, i.e., in red.

Here, the marker MK is comprised of a straight line portion L indicating the position of an extension line of the needle N. In the present example, the straight line portion L is a dashed line. The display image control section 53 locates the position of the extension line of the needle N in the ultrasonic image UI displayed in the display section 6 based on the information on a position detected by the first position locating section 51, and displays the marker MK at a corresponding position.

Next, at Step S5, the user inserts the needle N1 attached with the first magnetic sensor 10 into the subject. The user inserts the needle N1 while observing the B-mode image displayed in the display section 6. Once the needle N1 has been inserted at Step S5, the display image control section 53 displays a cross portion C indicating a needle tip of the needle N1 in the ultrasonic image UI according to the position of the needle tip, as shown in FIG. 5. The cross portion C constitutes the marker MK. When the cross portion C is displayed as part of the marker MK, the straight line portion L indicates the position of the extension line of the needle N, and besides, it indicates the position of the needle N1 per se. The display image control section 53 displays the marker MK based on the information on a position detected by the first position locating section 51.

Once the needle N1 has been inserted to reach a specified position at Step S5, the flow goes to processing at Step S6. At Step S6, the user makes an input at the operating section 7 for storing into the storage section 9 the information on a position of the needle N1 attached with the first magnetic sensor 10 and appended with the distinguishing information. For example, by the user pressing a specified button in the operating section 7 once, the control section 8 stores the information on a position into storage section 9.

The information on a position of the needle N1 is information on a position located by the first position locating section 51 for the needle N1 attached with the first magnetic sensor 10. The information on a position of the needle N1 is stored in the storage section 9 in connection with the color information appended to the needle N1.

Next, at Step S7, a decision is made as to whether or not storage of the distinguishing information and information on a position is completed. For example, at Step S7 here, an image for asking the user whether or not he/she will further perform storage of the distinguishing information and information on a position for another needle may be displayed in the display section 6. In the case that the user makes an input at the operating section 7 indicating that he/she will further perform storage of the distinguishing information and information on a position for another needle, the control section 8 decides that storage of the distinguishing information and information on a position is not completed (“NO” at Step S5), and the flow goes back to the processing at Step S2.

Returning from Step S5 to Step S2, the user removes the first magnetic sensor 10 from the needle N1, and attaches it to another needle N, as shown in FIG. 6. For example, the user attaches the first magnetic sensor 10 to the needle N2. In the present example, the number of the first magnetic sensor 10 is one, and the first magnetic sensor 10 is attached to a needle N whose distinguishing information and information on a position are to be stored.

At Step S3, once the button for storing the distinguishing information has been pressed, the control section 8 stores color information for differentiating the needle N2 attached with the first magnetic sensor 10 from the other needles N1, N3 in the storage section 9. In the case that an operation that blue is to be appended next to red is stored in the storage section 9, for example, color information appended to the needle N2 is blue. At Step S4, a marker MK in blue is displayed. Then, at Step S5, the needle N2 is inserted into the subject, and information on a position is stored at Step S5.

In the case that color information and information on a position for a needle N (needle N1) other than the needle N2 have been already stored in the storage section 9, the display image control section 53 may display the marker MK1 indicating the needle N1 along with the marker MK2 indicating the needle N2 in the ultrasonic image UI, as exemplarily shown in FIG. 7. This is the case in which information on a position stored in the storage section 9 exists in a cross section of the ultrasonic image UI whose information on a position is located by the second position locating section 52. In this case, the display image control section 53 displays the marker MK1 based on the color information and information on a position stored in the storage section 9. On the other hand, the marker MK2 is displayed based on the information on a position located by the first position locating section 51.

The processing at Steps from S2 to S7 is repeated until storage of the distinguishing information and information on a position is completed. In the present example, the processing at Steps from S2 to S7 is repeated until storage of the distinguishing information and information on a position for the needles N1, N2, N3 is completed. Therefore, when storage of the information on a position for the needle N2 has been performed at Step S6, it is decided that storage of the distinguishing information and information on a position is not completed at next Step S7, and the flow goes back to the processing at Step S2. Then, at Step S2 here, the user removes the first magnetic sensor 10 from the needle N2 and attaches it to the needle N3. Next, at Step S3, color information for differentiating the needle N3 from the other needles N1, N2 is stored in the storage section 9. In the case that an operation that yellow is to be appended next to blue is stored in the storage section 9, for example, color information to be appended to the needle N3 is yellow. At Step S4, a marker MK in yellow is displayed. Similarly, thereafter, the processing at Steps S5 and S6 is performed.

At Step S7, for example, in the case that the user makes an input indicating that storage of distinguishing information and information on a position is completed, the control section 8 decides that storage of distinguishing information and information on a position is completed (“YES” at Step S5), and terminates the processing.

In the case that a real-time ultrasonic image UI is displayed after the color information and information on a position for the three needles N1, N2, N3 have been stored in the storage section 9, markers MK indicating the needles N1, N2, N3 may be displayed in the ultrasonic image UI. Specifically, in the case that coordinates of the position of a needle N stored in the storage section 9 are included in an ultrasound transmission/reception plane whose information on a position is located by the second position locating section 52, the display image control section 53 displays a marker MK in the ultrasonic image UI. The color of the marker MK is the same as that in the color information stored in the storage section 9. The display image control section 53 displays the marker MK as a point in the ultrasonic image UI in the case that the ultrasound transmission/reception plane intersects the coordinates of the position of the needle N stored in the storage section 9.

Since the markers MK1, MK2, MK3 are displayed in different colors according to the present example, the user can easily know the correspondence between the actual needles N1, N2, N3 inserted into the subject and the markers MK1, MK2, MK3 displayed in the ultrasonic image UI. The needles N1, N2, N3 may be attached with labels in the same colors as those for the markers MK1, MK2, MK3.

Since the user can easily know the correspondence between the actual needles N1, N2, N3 and markers MK1, MK2, MK3, he/she can easily recognize which needle to move in the ultrasonic image UI when he/she wants to adjust the position of the needles N1, N2, N3.

Moreover, distinguishing information for a needle whose information on a position is to be stored may be appended in response to an input by the user at the operating section 7. In addition, the storage section 9 stores therein information on a plurality of different colors with their order of appending to the needles N specified and the user can append the information on a color merely by pressing a button on the operating section 7 once, so that the information on a color may be easily appended.

Furthermore, since the number of the first magnetic sensor 10 is only one, the number of cables for connecting the first magnetic sensor 10 with the apparatus main unit la may be reduced as compared with a case in which three first magnetic sensors 10 are respectively attached to the needles N1, N2, N3.

Next, a variation of the embodiment above will be described. First, a first variation will be described. In the embodiment above, an operation of appending the information on a color in an order of red, blue, and yellow is stored in the storage section 9; however, the order of appending the information on a color stored in the storage section 9 may be modified by the operating section 7 accepting an input from the user.

Next, a second variation will be described. In the case that a needle N moves after the information on a position of the needle N has been stored, the display image control section 53 may move a marker MK according to the movement of the needle N. Specifically, once the information on a position located by the first position locating section 51 has changed, the display image control section 53 moves a marker MK corresponding to one of the needles N1, N2, N3 whose information on positions is stored, wherein the one is attached with the first magnetic sensor 10 and its information on a position located by a position-detecting signal from the first magnetic sensor 10 matches the information on a position stored in the storage section 9. Thus, when the user adjusts the position of a needle N, its marker MK is moved, so that the user can easily confirm the position of the needle N.

Next, a third variation will be described. The distinguishing information is not limited to color information. For example, the distinguishing information may be numeric, character or symbol information. In such cases, the display image control section 53 displays a number, a character or a symbol along with a marker MK based on numeric, character or symbol information stored in the storage section 9. FIG. 8 shows a number N displayed along with a marker MK.

While the present invention has been described with reference to the embodiment, it will be easily recognized that the present invention may be practiced with several modifications without changing the spirit and scope thereof. For example, while the marker MK is not limited to that in the embodiment above.

We claim:

1. An ultrasonic diagnostic apparatus characterized in comprising:
a first position detecting section for detecting information on a position of a needle inserted into a subject in a specified coordinate system in a three-dimensional (3D) space;
an input section for accepting from a user inputs for, for a plurality of needles inserted into said subject, appending distinguishing information for distinguishing each of said plurality of needles and storing information on a position detected by said first position detecting section for a needle appended with said distinguishing information;
a second position detecting section for detecting information on a position in said 3D space of an ultrasound transmission/reception plane formed by an ultrasonic probe performing ultrasound transmission/reception to/from said subject; and
a display control section for displaying, in the case that a needle whose information on a position is stored is included in said ultrasound transmission/reception plane whose information on a position is detected by said second position detecting section, a marker indicating said needle in an ultrasonic image displayed in a display section for said transmission/reception plane in a manner that said needle is distinguishable from another needle based on said distinguishing information.

2. The ultrasonic diagnostic apparatus as recited in claim 1, characterized in comprising:
a storage section in which said plurality pieces of distinguishing information are stored with their order of appending to said needles specified, and
once said input section has accepted an input for appending said distinguishing information, said distinguishing information is appended in the order stored in said storage section.

3. The ultrasonic diagnostic apparatus as recited in claim 2, characterized in that: the order of appending said plurality pieces of distinguishing information stored in said storage section is modifiable by said input section accepting an input from the user.

4. The ultrasonic diagnostic apparatus as recited in claim 1, characterized in that:
said first position detecting section comprises one position sensor removably attached to said plurality of needles, and is for detecting information on a position of a needle attached with said position sensor, and
said input section accepts an input for storing information on a position detected for one of said plurality of needles that is attached with said position sensor.

5. The ultrasonic diagnostic apparatus as recited in claim 2, characterized in that:
said first position detecting section comprises one position sensor removably attached to said plurality of needles, and is for detecting information on a position of a needle attached with said position sensor, and
said input section accepts an input for storing information on a position detected for one of said plurality of needles that is attached with said position sensor.

6. The ultrasonic diagnostic apparatus as recited in claim 3, characterized in that:
said first position detecting section comprises one position sensor removably attached to said plurality of needles, and is for detecting information on a position of a needle attached with said position sensor, and
said input section accepts an input for storing information on a position detected for one of said plurality of needles that is attached with said position sensor.

7. The ultrasonic diagnostic apparatus as recited in claim 4, characterized in that:
in the case that the information on a position detected by said first detecting section has changed, said display control section moves a marker corresponding to one of said plurality of needles whose information on a position is stored, wherein said one is attached with said position sensor and its information on a position located by a position-detecting signal from said position sensor matches the stored information on a position.

8. The ultrasonic diagnostic apparatus as recited in claim 5, characterized in that:
in the case that the information on a position detected by said first detecting section has changed, said display control section moves a marker corresponding to one of said plurality of needles whose information on a position is stored, wherein said one is attached with said position sensor and its information on a position located by a position-detecting signal from said position sensor matches the stored information on a position.

9. The ultrasonic diagnostic apparatus as recited in claim 6, characterized in that:
in the case that the information on a position detected by said first detecting section has changed, said display control section moves a marker corresponding to one of said plurality of needles whose information on a position is stored, wherein said one is attached with said position sensor and its information on a position located by a position-detecting signal from said position sensor matches the stored information on a position.

10. The ultrasonic diagnostic apparatus as recited in claim 1, characterized in that:
the distinguishing information for distinguishing each of said plurality of needles is information on a color different from each other set for each of said plurality of markers, and
said display control section displays said markers in colors different from one another based on said information on a color.

11. The ultrasonic diagnostic apparatus as recited in claim 2, characterized in that:
the distinguishing information for distinguishing each of said plurality of needles is information on a color different from each other set for each of said plurality of markers, and
said display control section displays said markers in colors different from one another based on said information on a color.

12. The ultrasonic diagnostic apparatus as recited in claim 3, characterized in that:
the distinguishing information for distinguishing each of said plurality of needles is information on a color different from each other set for each of said plurality of markers, and
said display control section displays said markers in colors different from one another based on said information on a color.

13. The ultrasonic diagnostic apparatus as recited in claim 4, characterized in that:
the distinguishing information for distinguishing each of said plurality of needles is information on a color different from each other set for each of said plurality of markers, and
said display control section displays said markers in colors different from one another based on said information on a color.

14. The ultrasonic diagnostic apparatus as recited in claim 5, characterized in that:
the distinguishing information for distinguishing each of said plurality of needles is information on a color different from each other set for each of said plurality of markers, and
said display control section displays said markers in colors different from one another based on said information on a color.

15. The ultrasonic diagnostic apparatus as recited in claim 1, characterized in that:
the distinguishing information for distinguishing each of said plurality of needles is a piece of information different from each other set for each of said plurality of markers, said piece of information being of at least one of a number, a character, and a symbol displayed along with said marker, and
said display control section displays at least one of a number, a character, and a symbol different from each other along with said marker based on said information.

16. The ultrasonic diagnostic apparatus as recited in claim 2, characterized in that:
the distinguishing information for distinguishing each of said plurality of needles is a piece of information different from each other set for each of said plurality of markers, said piece of information being of at least one of a number, a character, and a symbol displayed along with said marker, and
said display control section displays at least one of a number, a character, and a symbol different from each other along with said marker based on said information.

17. The ultrasonic diagnostic apparatus as recited in claim 3, characterized in that:
the distinguishing information for distinguishing each of said plurality of needles is a piece of information different from each other set for each of said plurality of markers, said piece of information being of at least one of a number, a character, and a symbol displayed along with said marker, and
said display control section displays at least one of a number, a character, and a symbol different from each other along with said marker based on said information.

18. The ultrasonic diagnostic apparatus as recited in claim 4, characterized in that:
the distinguishing information for distinguishing each of said plurality of needles is a piece of information different from each other set for each of said plurality of markers, said piece of information being of at least one of a number, a character, and a symbol displayed along with said marker, and
said display control section displays at least one of a number, a character, and a symbol different from each other along with said marker based on said information.

19. The ultrasonic diagnostic apparatus as recited in claim 1, characterized in that:
the information on a position of said needle includes information on said needle per se and an extension line of said needle, and
said marker indicates said needle per se and the extension line of said needle.

20. An ultrasonic diagnostic apparatus comprising:
a processor; and
an input device, said apparatus being characterized in that:
said processor executes a first position detecting function, a second position detecting function, and a display control function by a program, wherein:
said first position detecting function is a function of detecting information on a position of a needle inserted into a subject in a specified coordinate system in a three-dimensional (3D) space;
said input device is for accepting from a user inputs for, for a plurality of needles inserted into said subject, appending distinguishing information for distinguishing each of said plurality of needles and storing information on a position detected by said first position detecting function for a needle appended with said distinguishing information;
said second position detecting function is a function of detecting information on a position in said 3D space of an ultrasound transmission/reception plane formed by an ultrasonic probe performing ultrasound transmission/reception to/from said subject; and
said display control function is a function of displaying, in the case that a needle whose information on a position is stored is included in said ultrasound transmission/reception plane whose information on a position is detected by said second position detecting function, a marker indicating said needle in an ultrasonic image displayed in a display section for said transmission/reception plane in a manner that said needle is distinguishable from another needle based on said distinguishing information.
